(19) United States
(12) Patent Application Publication
(10) Pub. No.: US 2009/0286883 A1
(43) Pub. Date: Nov. 19, 2009

(54) TREATMENT OF HEART FAILURE AND ASSOCIATED CONDITIONS BY ADMINISTRATION OF MONOAMINE OXIDASE INHIBITORS

(75) Inventors: Fabio DiLisa, Padova (IT); Ning Feng, Baltimore, MD (US); Nina Kaludercic, Baltimore, MD (US); Nazareno Paolocci, Baltimore, MD (US)

Correspondence Address: Swiss Tanner, P.C.;P.O. Box 1749, Four Main Street, Suite 100, Los Altos, CA, 94022, US

(73) Assignees: Nazareno Paolocci, Baltimore, MD (US); Universita' degli Studi di Padova (University of Padua), Padova (IT)

(21) Appl. No.: 12/407,739

(22) Filed: Mar. 19, 2009

Related U.S. Application Data
(60) Provisional application No. 61/038,230, filed on Mar. 20, 2008.
(60) Provisional application No. 61/155,704, filed on Feb. 26, 2009.

(51) Int. Cl. A61K 31/138; A61P 9/00

(52) U.S. Cl. 514/651

(57) ABSTRACT

Administration of monoamine oxidase inhibitors is useful in the prevention and treatment of heart failure and incipient heart failure.

### CROSS-REFERENCE TO RELATED PATENT APPLICATIONS

This application claims the benefit of U.S. Provisional Application Ser. No. 61/038,230 filed 20 Mar. 2008, and U.S. Provisional Application Ser. No. 61/155,704 filed 26 Feb. 2009, both of which applications are incorporated herein by reference in their entirety.

### STATEMENT REGARDING FEDERALLY SPONSORED RESEARCH OR DEVELOPMENT

This invention was made with government support under National Heart, Lung, and Blood Institute grant no. R01 HL075265-01A2. The government has certain rights in the invention.

### BACKGROUND OF THE INVENTION

1. Field of the Invention

The present invention relates generally to the field of medicine, pharmaceutical chemistry, biology and in particular to methods for treating and inhibiting heart failure and incipient heart failure.

2. State of the Art

In spite of various known treatment and preventive methods, heart failure continues to remain a major cause of world wide mortality. There is a strong need for new effective methods of therapy for prevention and treatment of heart failure and incipient heart failure. The present invention provides methods for preventing and treating heart failure and incipient heart failure by administering monoamine oxidase (MAO) inhibitors.

### SUMMARY OF THE INVENTION

This invention is directed towards novel methods for preventing and treating heart failure, incipient heart failure, and associated conditions, symptoms, signs, and disorders. In one aspect, the invention provides a method of treating heart failure in a mammal in need thereof, which method comprises administering to said mammal diagnosed with heart failure a therapeutically effective amount of an MAO inhibitor. In another aspect, the invention provides a method of preventing heart failure in a mammal in need thereof, which method comprises administering to said mammal diagnosed with a susceptibility to heart failure a therapeutically effective amount of a MAO inhibitor.

In another aspect, the invention provides a method of inhibiting incipient heart failure in a mammal in need thereof, which method comprises administering to said mammal diagnosed with incipient heart failure a therapeutically effective amount of a MAO inhibitor. In another aspect, the invention provides a method of treating incipient heart failure in a mammal in need thereof, which method comprises administering to said mammal diagnosed with incipient heart failure a therapeutically effective amount of a MAO inhibitor.

A variety of well known methods are useful for diagnosing heart failure or incipient heart failure, their signs and symptoms, and mammalian subject susceptible to such conditions. A variety of MAO inhibitors are useful for practicing the methods of the present invention. Also well known in the art is a variety of MAO inhibitors and their pharmaceutically acceptable compositions for administration, in accordance with the present invention, to mammals including, but not limited to, humans.

### BRIEF DESCRIPTION OF THE DRAWINGS

FIG. 1. Prevention of caspase-3 production from cardiomyocytes upon treatment with clorgyline.

FIG. 2. Interaction of MAO-A and MAO-B isoforms with neurotransmitters/dietary amines in the central and peripheral nervous system.

### DETAILED DESCRIPTION OF THE INVENTION

The detailed description of the various aspects and embodiments of the invention is divided into the following sections beginning with a definition of certain terms used herein.

### DEFINITIONS

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art. As used herein, certain terms may have the following defined meanings.

As used herein, the singular form “a,” “an” and “the” include singular and plural references unless the context clearly dictates otherwise.

As used herein, “compensated hypertrophy” refers to abnormal chamber function or enlargement of the heart coincident with an increase in muscle mass. For example, in cardiac disease, the compensation for the inefficiency of the heart's pump action is by enlisting the various reserves of the heart such as hypertrophy, enlargement, or increase in rate so as to maintain circulatory equilibrium and prevent the appearance of the signs of congestive heart failure.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

As used herein, “cardiomyocytes” or “cardiac myocytes” are specialized muscle cells which form the myocardium of the heart. Cardiomyocytes have five major components: cell membrane (sarcolemma) and T-tubules for impulse conduction; sarcoplasmic reticulum for contraction; contractile elements; mitochondria; and a nucleus.

As used herein, “heart failure (HF)” or “congestive heart failure (CHF)”, refers to a condition in which the heart can not pump enough blood to the body's other organs due to, for example, heart muscle malfunction, weakening of the heart muscle called cardiomyopathy, and other heart muscle related reasons. Congestive heart failure (CHF) is characterized, among other effects, by left ventricle (LV) chamber dilation, decreased LV contractility and elevated levels of circulating catecholamines. In another aspect, congestive heart failure occurs due to ischemic and other reperfusion, and other non-ischemic factors. Heart failure includes, but is not limited to, the following symptoms or signs: cardiac reperfusion injury, compensated hypertrophy, human end stage heart failure, hypertensive cardiomyopathy, left ventricular hypertension, left or right ventricular dilation, left or right ventricular failure, maladaptive hypertrophy, myocardial structural disarrangement (apoptosis and loss of cardiomyocyte) and myocardial dysfunction (loss in contraction and/or relaxation), and pressure overloaded heart.

As used herein, “incipient heart failure” refers to the early and/or mild appearance of symptoms, precursors or signs of heart failure. For example, and without limitation, humans and such other mammals suffering from incipient heart failure may show no symptoms of heart failure at rest and only mild symptoms of heart failure while exercising. However, incipient heart failure can lead to full fledged or end stage heart failure.

As used herein, “left ventricular hypertension (LVH)” is a condition wherein the cardiac muscle responds to increased resistance in the circulation by becoming enlarged. However, with time, the fibers of the hypertrophied heart muscle become thickened and shortened, and consequently less able to relax. Hypertension makes the myocardium work harder. The resulting hypertrophy is the product of the thickening and shortening of the muscle fibers of the heart. Under these conditions, it becomes more difficult for the heart to relax and go through the normal cycle of contraction and relaxation. Changes in the myocardium appear in the collagen resulting in increased stiffness. The outcome of this process is a heart that is less able to meet the output demands of normal circulation. There is an impaired diastolic relaxation, but also heightened vulnerability to ischemic events.

As used herein, “left ventricular dilation” refers to a left ventricular enlargement, which can increase the volume of blood that is ejected from the ventricle, temporarily improving cardiac output. This increase in size of the ventricular cavity, however, also results in a reduction of the percentage of the left ventricular volume of blood that is effected (called ejection fraction) and has significant physiological implications. “Left ventricular dilation” is a well-recognized precursor and sign of ventricular dysfunction and congestive heart failure after myocardial infarction. Similarly, “right ventricular dilation” refers to a right ventricular enlargement and associated signs or disorder.

As used herein, “left ventricular failure” refers to a disorder where the left side of the heart fails to pump blood effectively. This results in a back flow, pressure and/or congestion of blood into the lungs. Signs indicating “left ventricular failure” include a laterally displaced apex beat (which occurs if the heart is enlarged). A gallop rhythm (additional heart sounds) may be heard as a marker of increased blood flow, or increased intra-cardiac pressure. Similarly, “right ventricular failure” refers to a sign or disorder where the right side of the heart fails to pump blood effectively.

As used herein, “mammals” include, but are not limited to, murines, rats, simians, humans, farm animals, sport animals and pets.

As used herein, “monoamine oxidases” are enzymes that catalyze the oxidation of monoamines. Two monoamine oxidase (MAO) isoenzymes, MAO-A and MAO-B, are closely linked in opposite orientation on the X chromosome and are expressed in the outer mitochondrial membrane. In vivo, MAO-A and MAO-B oxidize monoamine neurotransmitters and dietary monoamines, the regulation of which is important in maintaining normal mental states. MAO-A prefers serotonin, norepinephrine, and dopamine as substrates, whereas MAO-B prefers phenylethylamine and trace amines. These proteins have been sequenced and characterized, see for example, the National Center for Biotechnology Information (NCBI) GenBank Accession Nos. for MAO-A gi|57284114|emb|CAI43120.1|[57284114]; gi|57209563|emb|CAI42421.1|[57209563]; gi|4557735|ref|NP_{—}000231.1|[4557735]; gi|54402320|gb|AAV34720.1|[54402320]; gi|54402314|gb|AAV34717.1|[54402314]; gi|54402302|gb|AAV34711.1 |[54402302]; gi|54402290|gb|AAV34705.1|[54402290]; gi|57284213|emb|CAI43216.1|[57284213] or gi|57209566|emb|CAI42424.1|[57209566] and the GenBank Accession Nos. for MAO-B gi|57209948|emb|CAI42522.1|[57209948]; gi|187376|gb|AAA59551.1|[187376]; gi|38202207|ref|NP_{—}000889.3|[38202207]; gi|57209564|emb|CAI42422.1|[57209564]; gi|57208148|emb|CAD92552.2|[57208148]; gi|18490291|gb|AAH22494.1|[18490291]; gi|553527|gb|AAB46386.1|[553527] or gi|187359|gb|AAA59550.1|[187359].

As used herein, “monoamine oxidase inhibitor” or “MAO inhibitor” refers to a compound that acts by inhibiting the activity of monoamine oxidase, including MAO-A and/or MAO-B. In one aspect, MAO inhibitors prevent the breakdown of monoamine neurotransmitters thereby increasing their in vivo availability. In another aspect, MAO inhibitors prevent the catabolism of dietary monoamines. In yet another aspect, MAO inhibitors prevent generation of reactive oxygen species (ROS). MAO inhibitors are well known in the art and are used for the treatment of neurodegenerative diseases. Examples of MAO-A inhibitors include, but are not limited to, Clorgyline, Minaprine, and the reversible MAO-A inhibitors Befloxatone, Brofaromine, Cimoxatone, Harmaline, Moclobemide, Pirlindole and Toloxatone. Examples of MAO-B inhibitors include, but are not limited to, Rasagiline, Selegiline and Pargyline. Examples of unselective MAO-A and MAO-B inhibitors include, but are not limited to, Iproclozide (Sursum), Iproniazid (Marsilid, Iprozid, Ipronid, Rivivol, Propilniazida), Isocarboxazid (Marplan), Mebanazine (Actomol), Metfendrazine (H.M.-11), Nialamide (Niamid), Phenelzine (Nardil), Pheniprazine (Catron), Phenoxypropazine (Drazine), Pivalylbenzhydrazine (Tersavid, Neomarsilid), Safrazine (Safra) and Tranylcypromine (Parnate). See, for example, Remington: The Science and Practice of Pharmacy, 21^{st} Edition, (2005, Lippincott Williams & Wilkins), pages 1517-1523, and Physicians' Desk Reference, Edition 60 (2006, Thomson PDR) page 1499 (each of which is incorporated herein by reference).

As used herein, “preventing” or “prevention” of a disease, disorder, symptom or condition means that the onset of the disease, disorder, symptom or condition in a mammal predisposed thereto is prevented such that the mammal does not manifest the disease, disorder, symptom or condition.

As used herein, a “therapeutically effective amount” or an “effective amount” is used synonymously with and intends an amount sufficient to effect beneficial or desired results. An effective amount can be administered in one or more administrations, applications, or dosages.

As used herein, “treating” or “treatment” of a disease, disorder, symptom or condition will depend on the disease, disorder, symptom or condition to be treated, and the mammal to be treated. In general, treatment intends one or more of inhibiting the progression of the manifested disease, disorder, symptom or condition as measured by clinical or sub-clinical parameters (where the term “inhibiting” or “inhibition” is intended to be a subset of “treating” or “treatment”), arresting the development of the disease, disorder, symptom or condition as measured by clinical or sub-clinical parameters, ameliorating or causing regression of the disease, disorder, symptom or condition as measured by clinical or sub-clinical parameters, or reducing pain or discomfort for the mammal treated as measured by clinical and/or pharmacological parameters. “Treating” does not include preventing the onset of the disease or condition.

### Monoamine Oxidase (MAO) and its Role in Heart Failure.

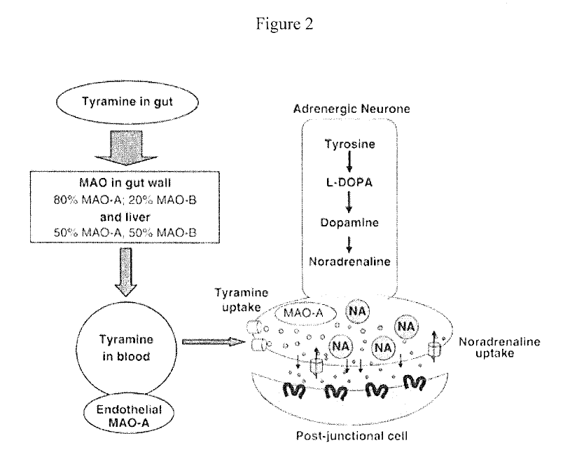

Monoamine oxidases (MAO) are flavoenzymes located within the outer mitochondrial membrane. MAOs are responsible for oxidative deamination of monoamino neurotransmitters and dietary monoamines¹. These exist in two different isoforms, MAO-A (FIG. 2) and MAO-B², sharing 70% sequence homology. These are distinguished by different substrate affinity and inhibitor sensitivity³.

MAO-A preferentially catalyses the oxidative deamination of norepinephrine (NE) and serotonin (5-HT) and is inhibited by low concentrations of clorgyline. MAO-B has affinity for phenylethylamine and benzylamine, and is inhibited by selegiline. Both isoforms catalyze the deamination of dopamine, tyramine, octopamine and tryptamine and are inhibited by pargyline⁴. Deletion of MAO-A and MAO-B genes has proven their role in neurotransmitter metabolism and behavior.

MAO-A null mice have elevated brain levels of 5-HT, NE, and, to a lesser extent, dopamine. 2-Phenylethylamine is increased in MAO-B knock out (KO) mice⁴. Compulsive-aggressive behavior results from lack of MAO-A function both in humans⁵ and mice⁶. It appears that MAO-A is active during development since this effect can be mimicked by the administration of MAO inhibitors during the early postnatal period. However, these studies report that MAO may not be essential for survival⁷.

The role of MAO in terminating the actions of neurotransmitters/dietary amines in central and peripheral nervous system (and in the extraneuronal tissue) has been reported⁸. MAO inhibitors are used for treating neurodegenerative diseases and affective disorders. MAO-A inhibitors are particularly effective in the treatment of depression by increasing brain levels of dopamine, NE and 5-HT. MAO-B inhibitors have been used for treating patients with Parkinson's and Alzheimer's disease.

In contrast, less attention has been given to the byproducts of MAO's monoamine catabolism. The monoamine catabolism byproducts generated by MAO are aldehydes, ammonia and H₂O₂. H₂O₂ is a toxic, reactive oxygen species,⁹⁻¹¹ or can generate toxic hydroxyl radicals in the presence of Fe²⁺.

In the heart, MAO-A is the prevalent isoform, and the majority of the enzyme is situated in the cardiomyocytes¹². In the peripheral tissues, MAO protects the body by oxidizing blood-derived amines or by preventing their entry into the bloodstream⁴ (FIG. 2).

### Therapies

The present invention relates in part to the discovery that MAO-A and MAO-B have major implications in the progression of compensated hypertrophy to left ventricular dilation and failure. Accordingly, the present invention provides that inhibition of MAOs, such as MAO-A, effectively prevents or retards this progression, improves myocardial function, and may also prevent loss of viable myocardial cells due to increased oxidative stress and pro-apoptotic signaling.

In one aspect, the invention provides a method of treating heart failure in a mammal in need thereof, which method comprises administering to said mammal diagnosed with heart failure a therapeutically effective amount of an MAO inhibitor. In another aspect, the invention provides a method of preventing heart failure in a mammal in need thereof, which method comprises administering to said mammal diagnosed with a susceptibility to heart failure a therapeutically effective amount of a MAO inhibitor.

In another aspect, the invention provides a method of inhibiting incipient heart failure in a mammal in need thereof, which method comprises administering to said mammal diagnosed with incipient heart failure a therapeutically effective amount of a MAO inhibitor. In another aspect, the invention provides a method of treating incipient heart failure in a mammal in need thereof, which method comprises administering to said mammal diagnosed with incipient heart failure a therapeutically effective amount of a MAO inhibitor.

In certain embodiments, the MAO-A (or -B) inhibitors administered in accordance with the invention are those, which, when administered, do not give rise to the potentially harmful side-reaction called “cheese-effect.” Cheese-effect refers to hypertensive crises in patients under treatment with MAO inhibitors following the ingestion of food rich in tyramine (chocolate, cheese, etc)¹⁶ decompensated, maladaptive cardiac hypertrophy and functional failure. Thus, in certain embodiment, the MAO inhibitors administered in accordance with the present invention exclude irreversible MAO inhibitors that cause “cheese-effect.” In certain other embodiments, the MAO inhibitors administered in accordance with the present invention are the reversible MAO-A or RIMA inhibitors. Such MAO inhibitors are well known to one of skill in the art.

In certain embodiments, the methods of the invention exclude methods of preventing postischemic oxidative stress and myocyte hypertrophy. In certain other embodiments, the methods of the invention include methods of preventing postischemic oxidative stress and myocyte hypertrophy.

The invention having been described in summary, in detail, and by the accompanying figure, is exemplified, and not limited, by the following examples which demonstrate the usefulness of administering MAO inhibitors for the prevention and treatment of heart failure, incipient heart failure, and one or more of their signs and symptoms.

### EXAMPLES

The practice of the present invention employs, unless otherwise indicated, conventional techniques of molecular biology (including recombinant techniques), microbiology, cell biology, biochemistry, pharmacology, immunology, and chemistry, which are well within the skill of on of art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); CULTURE OF ANIMAL CELLS: A MANUAL OF BASIC TECHNIQUE (R. I. Freshney 5^{th} edition (2005)); and GOOMAN AND GILLMAN'S THE PHARMACOLOGICAL BASIS OF THERAPEUTICS (Brunton et al. McGraw Hill Publishing (2005)).

### 1. In Vivo Administration of an MAO Inhibitor Prevents Cardiac Decompensation After 6 Weeks of Transverse Aortic Constriction (TAC).

MAO as a source of ROS in cardiac reperfusion injury¹³ and its role in in vitro pro-hypertrophic effects of serotonin has been explored¹⁴. In this experiment, MAO's expression, activity, and role in congestive heart failure (CHF) was tested in vivo. The MAO-A gene expression was 3.5-fold higher in C57B16 mice after 6 weeks of TAC-induced pressure overload (T6W, n=6, Table 1). TAC-induced pressure overload is a condition associated with chamber dilation, reduced basal and beta-stimulated contractility and depressed overall LV function.

TABLE 1
MAO-A gene expression in mouse hearts after 6 weeks of TAC.

| | MAO-A/GAPDH |
|---|---|
| Sham | 0.27 ± 0.05 |
| TAC 6 weeks | 0.96 ± 0.22 * |

* p < 0.05 vs sham

The effects of MAO-A inhibition under pharmacological conditions on the left ventricular hypertension (LVH) development were also tested. As shown in Table 2, after 3 weeks of TAC, wall thickness was increased in saline- and clorgyline-treated mice (CLO, 1 mg/kg/day, n=6) when compared to sham operated animals, likely reflecting a compensation for the increase in pressure. However, LV function was significantly better in CLO-treated group (CLO-mice): both fractional shortening and ejection fraction were comparable to values reported for control mice (sham). At 6 weeks of TAC, differences between the two groups became even more pronounced. The CLO-mice displayed improved LV function: fractional shortening was 30±5.4 vs 61±0.8%, and ejection fraction 60.2±8.2 vs 93.6±0.4% (Table 2).

TABLE 2
Time-dependent changes in cardiac morphology and in vivo ventricular function
induced by TAC in control and clorgyline treated mice.

| | Sham | T3 w | T3 w + CLO | T6 w | T6 w + CLO |
|---|---|---|---|---|---|
| BW (g) | 27.2 ± 1.3 | 25.4 ± 0.6 | 25.6 ± 0.6 | 26.6 ± 0.7 | 27.1 ± 0.4 |
| Heart rate (bpm) | 738 ± 11.2 | 670 ± 11^{A} | 702 ± 12 | 627.5 ± 24^{A} | 670 ± 13.7 |
| IVS (mm) | 0.8 ± 0.02 | 1.3 ± 0.04^{B} | 1.3 ± 0.02^{B} | 1.2 ± 0.04 | 1.4 ± 0.03^{B} |
| LVEDD (mm) | 3.2 ± 0.02 | 2.9 ± 0.2 | 2.9 ± 0.06 | 4.0 ± 0.2 | 3.0 ± 0.08^{D} |
| LVESD (mm) | 1.2 ± 0.02 | 1.7 ± 0.2 | 1.2 ± 0.06 | 2.9 ± 0.4^{B} | 1.2 ± 0.04^{D} |
| LVPW (mm) | 0.8 ± 0.02 | 1.3 ± 0.04^{B} | 1.2 ± 0.03^{B} | 1.1 ± 0.05^{B} | 1.3 ± 0.03^{B, D} |
| FS (%) | 63.5 ± 0.9 | 43.9 ± 3.2^{B} | 56.8 ± 1.9^{C} | 30.6 ± 5.3^{B} | 61.6 ± 0.9^{D} |
| EF (%) | 94.7 ± 0.4 | 81 ± 7.9^{A} | 91.6 ± 0.4^{C} | 62.4 ± 7.9^{B} | 94.3 ± 0.4^{D} |
| LV mass/BW (mg/g) | 2.8 ± 0.1 | 6.9 ± 0.4^{B} | 6.4 ± 0.3^{B} | 9.3 ± 0.5^{B} | 7.2 ± 0.4^{B, D} |

IVS: interventricular septum,
LVEDD: left ventricular end-diastolic dimension,
LVESD: left ventricular end-systolic dimension,
LVPW: left ventricular posterior wall,
FS: fractional shortening,
EF: ejection fraction,
BW: body weight,
T3 w: TAC 3 weeks,
T6 w: TAC 6 weeks,
CLO: clorgyline.
^{A}p < 0.05 vs sham,
^{B}p < 0.001 vs sham,
^{C}p < 0.05 vs TAC 3 weeks,
^{D}p < 0.05 vs TAC 6 weeks.

Moreover, the CLO-mice showed reduced levels of hypertrophy: LV mass, left ventricular end diastolic dimension (LVEDD), and end systolic dimension (LVESD) were all significantly reduced (Table 2). These results demonstrate that inhibiting MAO-A by administering a therapeutically effective amount of an MAO inhibitor prevents or reduces maladaptive LV hypertrophy, chamber dilation, and LV dysfunction in pressure-overloaded hearts, and is thus useful in practicing the methods of the invention.

2. Administration of the MAO-A Inhibitor Clorgyline Results in Reduced Levels of Apoptosis in heart cells after 6 weeks of TAC.

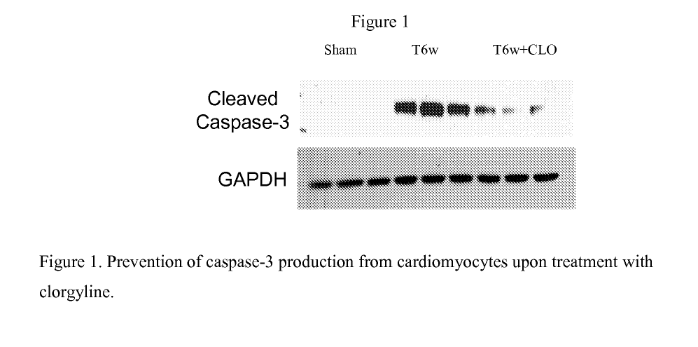

The MAO-A inhibitor clorgyline impacted the pro-apoptotic signaling cascade as demonstrated below. Levels of cleaved caspase-3 were markedly elevated after 6 weeks in TAC mice vs shams. Clorgyline treatment prevented caspase-3 activation demonstrating that loss of viable cardiomyocytes is a major contributor to impaired ventricular function and onset of chamber dilation (FIG. 1).

### 3. MAO-A Expression is Induced by Pro-Hvpertrophic Stimuli and the Up-Regulation of MAO-Atriggers Hypertrophic Signaling.

TABLE 3
MAO-A and BNP gene expression after treatment with
NE in the absence or presence of clorgyline.

| | MAO-A | BNP |
|---|---|---|
| | (vs GAPDH) | (vs GAPDH) |
| control | 0.58 ± 0.05 | 1 ± 0 |
| NE | 1.85 ± 0.04 * | 8.9 ± 1.4 * |
| NE + | — | 4.85 ± 0.9 * ^{#} |
| clorgyline | | |

* p < 0.05 vs control,
^{#} p < 0.05 vs NE.

This experiment demonstrates a causative link between pro-hypertrophic agents and MAO-A. Incubation of neonatal rat cardiomyocytes with NE (10 μM) for 24 hrs resulted in up-regulation of MAO-A gene expression and induction of hypertrophy, as measured by an increase in brain natriuretic peptide (BNP) expression (Table 3). MAO-A inhibition partially reduced NE induced hypertrophy, suggesting that this hormone is transported into the cell and degraded by MAO-A while H₂O₂ is produced. This process may contribute to the development of

TABLE 4
EMT gene expression in mouse hearts upon stimulation with tyramine.

| | EMT/GAPDH |
|---|---|
| control | 0.5 ± 0.04 |
| tyramine | 0.97 ± 0.2 * |

* p < 0.05 vs control.

hypertrophy. Exposing myocytes to another MAO substrate, tyramine (10 μM), resulted in a 2-fold increase in EMT gene expression (Table 4), the main monoamine transporter present at the cardiomyocyte level. A direct link between MAO-A activity and myocyte hypertrophy was further corroborated by the fact that tyramine incubation of cardiomyocytes resulted in 2-fold increase of NFAT4 expression, a transcription factor involved in maladaptive hypertrophy (Table 5).

TABLE 5
NFAT4 gene expression (normalized to GAPDH) upon stimulation
with tyramine in the absence or presence of clorgyline.

| | −clorgyline | +clorgyline |
|---|---|---|
| Control | 0.24 ± 0.03 | 0.23 ± 0.02 |
| Tyramine | 0.38 ± 0.01 * | 0.32 ± 0.02 ^{#} |

* p < 0.05 vs control,
^{#} p < 0.05 vs tyramine.

Preincubation with clorgyline prior to tyramine treatment reduced this effect significantly.

These results demonstrate that MAO activity in the heart can trigger the pro-hypertrophic response via activation of pathways promoting maladaptive hypertrophy and cause heart failure; administration of an MAO-A inhibitor reduces these symptoms, and is therapeutically beneficial.

### 4. MAO-A is a Major Source of Reactive Oxygen Species (ROS) and Inhibition of MAO-A Prevents Oxidative Stress in Cardiomyocytes.

This example demonstrates that the administration of MAO inhibitors prevents oxidative stress in cardiomyocytes and is useful in preventing and treating heart failure or an associated symptom or condition in accordance with the invention. In vivo, mitochondria and the respiratory chain, in particular, are considered a major intracellular ROS source¹⁵. MAOs are located in the outer membrane of these organelles and MAO-A can increase oxidative burden. When HL-1 cardiomyocytes were incubated with 100 μM H₂O₂ or 5 μM arachidonic acid, a rise in ROS production at mitochondrial level was observed (Table 6).

TABLE 6
Levels of oxidative stress upon stimulation with
H₂O₂ or arachidonic acid in the absence or presence of
deprenyl, clorgyline or in siRNA treated cells.

| | H₂O₂ | Arachidonic acid |
|---|---|---|
| | (% increase vs | (% increase vs |
| | control) | control) |
| No treatment | 156.2 ± 4.3 | 145.6 ± 3.8 |
| +deprenyl | 150.7 ± 4.9 | 142.4 ± 0.3 |
| +clorgyline | 122.6 ± 3 * | 98.3 ± 1.3 * |
| +MAO-A siRNA | 122.3 ± 0.4 * | 99 ± 2.7 * |

* p < 0.05 vs no treatment.

The rise in ROS production was completely prevented, or reduced, by cell pretreatment with clorgyline (1 μM). No protection was observed with MAO-B inhibitor selegiline. These results were also confirmed in siRNA treated cells in which MAO-A expression was reduced by 90%.

### REFERENCE LIST

(1) Kumar M J, Nicholls D G, Andersen J K. Oxidative alpha-ketoglutarate dehydrogenase inhibition via subtle elevations in monoamine oxidase B levels results in loss of spare respiratory capacity: implications for Parkinson's disease. J Biol Chem 2003 Nov. 21; 278(47):46432-9.
(2) Shih J C, Chen K. Regulation of MAO-A and MAO-B gene expression. Curr Med Chem 2004 August; 11(15):1995-2005.
(3) Youdim M B, Finberg J P. New directions in monoamine oxidase A and B selective inhibitors and substrates. Biochem Pharmacol 1991 Jan. 15; 41(2):155-62.
(4) Youdim M B, Edmondson D, Tipton K F. The therapeutic potential of monoamine oxidase inhibitors. Nat Rev Neurosci 2006 April; 7(4):295-309.
(5) Brunner H G, Nelen M, Breakefield X O, Ropers H H, van Oost B A. Abnormal behavior associated with a point mutation in the structural gene for monoamine oxidase A. Science 1993 Oct. 22; 262(5133):578-80.
(6) Shih J C. Cloning, after cloning, knock-out mice, and physiological functions of MAO-A and B. Neurotoxicology 2004 January; 25(1-2):21-30.
(7) Lenders J W, Eisenhofer G, Abeling N G et al. Specific genetic deficiencies of the A and B isoenzymes of monoamine oxidase are characterized by distinct neurochemical and clinical phenotypes. J Clin Invest 1996 Feb. 15; 97(4):1010-9.
(8) Billett E E. Monoamine oxidase (MAO) in human peripheral tissues. Neurotoxicology 2004 January; 25(1-2): 139-48.
(9) Yang L, Omori K, Suzukawa J, Inagaki C. Calcineurin-mediated BAD Ser155 dephosphorylation in ammonia-induced apoptosis of cultured rat hippocampal neurons. Neurosci Lett 2004 Feb. 26; 357(1):73-5.
(10) Halliwell B. Reactive oxygen species and the central nervous system. J Neurochem 1992 November; 59(5):1609-23.
(11) Lamensdorf I, Eisenhofer G, Harvey-White J, Hayakawa Y, Kirk K, Kopin I J. Metabolic stress in PC12 cells induces the formation of the endogenous dopaminergic neurotoxin, 3,4-dihydroxyphenylacetaldehyde. J Neurosci Res 2000 May 15; 60(4):552-8.
(12) Partanen S, Penttila O, Kyosola K, Merikallio E, Siltanen P. Histochemically demonstrable monoamine oxidase activity in the adult human heart in various cardiac diseases. Virchows Arch A Pathol Anat Histol 1976 Jul. 21; 370(4):291-6.
(13) Bianchi P, Kunduzova O, Masini E et al. Oxidative stress by monoamine oxidase mediates receptor-independent cardiomyocyte apoptosis by serotonin and postischemic myocardial injury. Circulation 2005 Nov. 22; 112(21):3297-305.
(14) Bianchi P, Pimentel D R, Murphy M P, Colucci W S, Parini A. A new hypertrophic mechanism of serotonin in cardiac myocytes: receptor-independent ROS generation. FASEB J 2005 April; 19(6):641-3.
(15) Di Lisa, Canton M, Menabo R, Kaludercic N, Bernardi P. Mitochondria and cardioprotection. Heart Fail Rev 2007 December; 12(3-4):249-60.
(16) Youdim M B, Weinstock M Therapeutic applications of selective and non-selective inhibitors of monoamine oxidase A and B that do not cause significant tyramine potentiation. Neurotoxicology. 2004 January; 25(1-2):243-50.

Throughout this disclosure, various publications, patents and/or published patent specifications are referenced by an identifying citation. The disclosures of these publications, patents and published patent specifications are hereby incorporated by reference into the present disclosure to more fully describe the state of the art to which this invention pertains.

What is claimed is:

1. A method of treating heart failure in a mammal in need thereof, which method comprises administering to said mammal diagnosed with heart failure a therapeutically effective amount of a monoamine oxidase inhibitor.

2. A method of inhibiting incipient heart failure in a mammal in need thereof, which method comprises administering to said mammal diagnosed with incipient heart failure a therapeutically effective amount of a monoamine oxidase inhibitor.

3. A method of treating incipient heart failure in a mammal in need thereof, which method comprises administering to said mammal diagnosed with incipient heart failure a therapeutically effective amount of a monoamine oxidase inhibitor.
